(19) United States
(12) Patent Application Publication
(10) Pub. No.: US 2009/0269733 A1
(43) Pub. Date: Oct. 29, 2009

(54) Devices and Methods for the Rapid Analysis of Pathogens in Biological Fluids

(75) Inventor: Waheed N. Khan, North Bethesda, MD (US)

Correspondence Address: EDELL, SHAPIRO & FINNAN, LLC, 1901 RESEARCH BLVD., SUITE 400, ROCKVILLE, MD, 20850-3164, US

(73) Assignee: AZKO, INC., N. Bethesda, MD (US)

(21) Appl. No.: 12/426,362

(22) Filed: Apr. 20, 2009

Related U.S. Application Data
(60) Provisional application No. 61/047,350, filed on Apr. 23, 2008.

(51) Int. Cl. C12Q 1/70; C12M 1/34; C12Q 1/04

(52) U.S. Cl. 435/5; 435/287.1; 435/287.2; 435/34

(57) ABSTRACT

The present invention relates to devices and methods for rapidly determining whether a biological fluid contains a suspect Gram positive bacterial, a Gram negative bacterial or a viral pathogen. The invention particularly pertains to such devices and methods wherein the biological fluid is cerebrospinal fluid, and wherein the suspect pathogen is a causative agent of meningitis.

### CROSS-REFERENCE TO RELATED APPLICATIONS

This application claims priority to U.S. Patent Application Ser. No. 61/047,350 (filed Apr. 23, 2008; pending), which application is herein incorporated by reference in its entirety.

### BACKGROUND OF THE INVENTION

1. Field of the Invention

The present invention relates to devices and methods for rapidly determining whether a biological fluid contains a suspect Gram positive bacterial, a Gram negative bacterial or a viral pathogen. The invention particularly pertains to such devices and methods wherein the biological fluid is cerebrospinal fluid, and wherein the suspect pathogen is a causative agent of meningitis.

2. Description of Related Art

The prompt and accurate diagnosis of the causative agents of human disease represent critical goals of modern medical treatment. In many cases, attempting to directly isolate microorganisms from human tissue samples is not feasible due to extended culturing times, fastidious nutritional and culturing requirements, or fast-moving disease progression.

Meningitis is a disease of particular concern. Meningitis is an inflammation of the meninges, which are the thin layers of tissue that cover the brain and the spinal cord. Meningitis is most commonly caused by infection (by bacteria, viruses, or fungi). The most dangerous forms of meningitis are those caused by bacteria. The bacterial form of meningitis is an extremely serious illness that requires immediate medical care. If not treated quickly, it can lead to death within hours; it results in permanent brain damage in about 30% of people. Three species of bacteria account for most cases of acute bacterial meningitis: Neisseria meningitidis, Haemophilus influenzae type b and Streptococcus pneumoniae. Together, these three bacteria account for about 80% of bacterial meningitis cases in the U.S. (Fitch, M. T. et al. (2007) “Emergency Diagnosis and Treatment of Adult Meningitis,” Lancet Infect. Dis. 7:191-200; Mace, S. E. et al. (2008) “Acute Bacterial Meningitis,” Emer. Med. Clin. N. Am. 38:281-317). These organisms are normally present in the external environment and may reside in the upper respiratory system without causing harm (see, CELLULAR AND MOLECULAR IMMUNOLOGY, Abbas, Lichtman, Pober, Eds. 2000, 4^{th} Ed.).

In some cases, infection develops because the immune system is impaired—as it is in people who have an HIV (human immunodeficiency virus) infection. Infection may also result from a head injury. A skull fracture may create an opening between the nasal sinuses and the space around the meninges (which contains cerebrospinal fluid). Bacteria can travel from the sinuses through the opening and infect the meninges (see, CELLULAR MICROBIOLOGY, Cossart, Boquet, Normark, Rappuoli, Eds. 2000, ASM Press). People most at risk of developing meningitis due to Neisseria meningitidis and Streptococcus pneumoniae are those who abuse alcohol; those who have had a splenectomy (removal of the spleen); those who have chronic infections of the middle ear, nose, or sinuses; and those who have pneumococcal pneumonia or sickle cell disease. Listeria monocytogenes causes about 10% of cases of bacterial meningitis. People who have kidney failure or who are taking corticosteroids (which suppress the immune system) have a higher-than-average risk of developing meningitis due to Listeria bacteria. Other types of bacteria can also cause meningitis. Meningitis due to Escherichia coli (found normally in the colon and in feces) or Klebsiella bacteria usually develops after a head injury, brain or spinal cord surgery, a widespread infection of the blood (sepsis), or an infection acquired in a hospital. These infections are more common among people with an impaired immune system. Newborns, whose immune system are not completely formed, are at increased risk of developing infections due to Escherichia coli or group B Streptococci.

Viral meningitis, often called encephalitis, is more common than the bacterial form and generally less serious. Enteroviruses (of the family Picornaviridae, e.g., echoviruses, coxsackieviruses A and B, polioviruses, non-polio enteroviruses and the numbered enteroviruses) account for more than 85% of all cases of viral meningitis. The overwhelming majority of viral meningitis cases are caused by serotypes of coxsackie and echoviruses. Coxsackievirus B subgroups alone account for more than 60% of meningitis cases in children younger than 3 months. Arboviruses (e.g., eastern and western equine encephalitis viruses, St. Louis encephalitis, West Nile, Japanese B, and Murray Valley, etc.) account for about 5% percent of cases in North America. The mumps and measles viruses can cause meningitis, however, due to vaccination in developed countries, mumps virus is a significant cause of meningitis (10-20% of cases) only in developing areas of the world where vaccines are not readily accessible; meningitis caused by measles is rare. Herpes family viruses (HSV-1, HSV-2, VZV, EBV, CMV, and human herpesvirus 6) collectively cause approximately 4% of cases of viral meningitis, with HSV-2 being the most common causative agent. In rare instances, lymphocytic choriomeningitis virus infection can cause meningitis. Adenovirus is a rare cause of meningitis in immunocompetent individuals but a major cause in AIDS patients. Similarly, HIV may be a cause of meningitis. Reports have suggested that as many as 5-10% of HIV infections can be heralded by meningitis. Viral meningitis is reviewed by Logan, S. A. et al. (2008) (“Viral Meningitis,” Brit. Med. J. 336(7634):36-40); Chadwick, D. R. (2006) (“Viral Meningitis,” Brit. Med. Bull. 75-76:1-14); Leite, C. et al. (2005) (“Viral Diseases Of The Central Nervous System,” Top. Magn. Reson. Imaging 16(2):189-212); Kimmig, P. et al. (2002) (“Enteroviruses—Again And Again The Cause Of Serous Meningitis,” Dtsch. Med. Wochenschr. 127(49):2604); and Sawyer, M. H. (2002) (“Enterovirus Infections: Diagnosis And Treatment,” Semin. Pediatr. Infect. Dis. 13(1):40-47).

In order to correctly diagnose meningitis, whether bacterial or viral, a lumbar puncture is required to obtain a sample of the patient's cerebrospinal fluid (CSF). Nucleic amplification techniques (such as the polymerase chain reaction (Mullis, K. et al., “Specific Enzymatic Amplification of DNA in Vitro: The Polymerase Chain Reaction,” Cold Spring Harbor Symp. Quant. Biol. 51:263-273 (1986); Higuchi, R. “PCR Technology,” Ehrlich, H. (ed.), Stockton Press, NY, 1989, pp 61-68; EP 50,424; EP 84,796, EP 258,017, EP 237,362; EP 201,184; U.S. Pat. Nos. 4,683,202; 4,582,788; 4,683,194), rolling circle amplification (U.S. Pat. Nos. 5,354,668; 5,591,609; 5,614,389; 5,733,733; 5,834,202; 5,854,033; 6,124,120; 6,143,495; 6,183,960; 6,210,884; 6,218,152; 6,261,808; 6,280,949; 6,287,824; 6,344,329; 6,448,017; 6,740,745) and other amplification technologies (Kwoh D. et al., “Transcription-Based Amplification System and Detection of Amplified Human Immunodeficiency Virus Type 1 with a Bead-Based Sandwich Hybridization Assay,” Proc. Natl. Acad. Sci. (U.S.A.) 86:1173 (1989); Wu, D. Y. et al., “The Ligation Amplification Reaction (LAR)—Amplification of Specific DNA Sequences Using Sequential Rounds of Template-Dependent Ligation,” Genomics 4:560 (1989); Walker, G. T. et al., “Isothermal in vitro Amplification of DNA by a Restriction Enzyme/DNA Polymerase System,” Proc. Natl. Acad. Sci. (U.S.A.) 89:392-396 (1992); U.S. Pat. Nos. 5,270,184; 5,455,166) have been proposed for use in detecting Mycobacterium tuberculosis, herpes simplex virus, enteroviruses, cytomegalovirus and Toxoplasma gondii (Fitch, M T et al. (2007) “Emergency Diagnosis and Treatment of Adult Meningitis,” Lancet Infect. Dis. 7:191-200; Lorino, G. et al. (2000) “Diagnostic Values Of Cytokine Assays In Cerebrospinal Fluid In Culture-Negative, Polymerase Chain Reaction-Positive Bacterial Meningitis,” Eur. J. Clin. Microbiol. Infect. Dis. 19:388-392). However, despite the promise of such approaches, the diagnosis of pathogens, and especially of pathogens of the CSF remains problematic (Michelow, I. C. et al. (2000) “Value Of Cerebrospinal Fluid Leukocyte Aggregation In Distinguishing The Causes Of Meningitis In Children,” Pediatr. Infect. Dis. J. 19(1):66-72).

Accordingly, despite the availability of such tests, meningitis remains typically determined by testing the CSF to detect the presence of bacteria or blood, as well as to measure glucose levels (a low glucose level is indicative of bacterial or fungal meningitis (see, Mace, S. E. et al. (2008) “Acute Bacterial Meningitis,” Emer. Med. Clin. N. Am. 38:281-317; Negrini, B. et al. (2000) “Cerebrospinal Fluid Findings In Aspetic Versus Bacterial Meningitis,” Pediatrics 105(2): 316-319), lactate and white blood cell counts. In bacterial and cryptococcal infection, an increase in CSF lactate levels is found earlier than a reduced glucose. In viral meningitis, lactate levels remain normal, even when neutrophils are present in the CSF. Raised levels may also occur with severe cerebral hypoxia or genetic lactic acidosis. (Negrini, B. et al. “Cerebrospinal Fluid Findings In Aspetic Versus Bacterial Meningitis,” 2000, Pediatrics 105(2): 316-319; Van Acker, J. T. et al. “Automated Flow Cytometric Analysis Of Cerebrospinal Fluid,” 2001, Clinical Chem. 47(3): 556-560). Additional diagnostic procedures include performing a count of white blood cells. The white cell count is increased when there is inflammation of the central nervous system, particularly the meninges. Bacterial infections (e.g. meningitis, cerebral abscess, early tuberculous meningitis, septicaemia) are usually associated with the presence of neutrophils in the CSF. Viral infections are associated with an increase in mononuclear cells, although in some (e.g., Coxsackievirus, poliovirus infection) there may be an early increase in neutrophils. An increase in both neutrophils and mononuclear cells occurs in tuberculous meningitis and early viral meningitis. Eosinophils are seen in meningitis caused by Angiostrongylus cantonensis and in cysticercosis and coccidioidomycosis. Red cells are present HSV encephalitis (Negrini, B. et al. “Cerebrospinal Fluid Findings In Aspetic Versus Bacterial Meningitis,” 2000, Pediatrics 105(2): 316-319; Van Acker, J. T. et al. “Automated Flow Cytometric Analysis Of Cerebrospinal Fluid,” 2001, Clinical Chem. 47(3): 556-560; Dubos, F. et al. “Clinical Decision Rules To Distinguish Between Bacterial And Aseptic Meningitis, 2006, Arch. Dis. Child 91:647-650).

The Gram stain is a century-old empirical method for differentiating bacterial species based on the chemical and physical properties of bacterial cell walls (Gram, H. C. (1884). “Über die isolierte Färbung der Schizomyceten in Schnitt- und Trockenpräparaten,” Fortschritte der Medizin 2:185-189; Beveridge, T. J. (2001) “Use Of The Gram Stain In Microbiology,” Biotech. Histochem. 76(3):111-118; Yamanaka, K. (2002) “The Gram Stain,” Rinsho Biseibutshu Jinsoku Shindan Kenkyukai Shi. 12(2):81-90; Popescu, A. et al. (1996) “The Gram Stain After More Than A Century,” Biotech. Histochem. 71(3):145-151; Beveridge, T. J. (1990) “Mechanism Of Gram Variability In Select Bacteria,” J. Bacteriol. 172(3): 1609-1620). Bacteria that yield a positive report (“Gram positive bacteria) have a thick mesh-like cell wall made of peptidoglycan (50-90% of cell wall). Bacteria that yield a negative report (“Gram negative bacteria) have an additional lipid-containing, outer membrane that is separated from the cell wall by the periplasmic space. As is well known, the Gram staining method entails using heat to fix a bacterial specimen to a glass slide. The specimen is then subjected to staining with crystal violet (2 g of 90% crystal violet dissolved in 20 ml of 95% ethyl alcohol) (thereby staining all bacterial cells dark blue-violet). Gram's iodine (1 g of iodine, 2 g of potassium iodide per 300 ml of distilled water) is then applied to the specimen and serves to fix the crystal violet to the bacterial cell wall. The specimen is then washed with 50% ethyl alcohol, 50% acetone, which serve as decolorizers. If the bacteria is Gram-positive it will retain the primary stain and appear dark blue-violet; if it is Gram-negative it will lose the primary stain and appear colorless. To improve contrast, the decolorized specimen is treated with a secondary stain (typically safranin). Safranin does not appreciably alter the dark color of the Gram-positive bacteria, but renders the formerly colorless Gram negative bacteria red-pink. Properly performed, the Gram stain, differentiates nearly all bacteria into two major groups. The Gram-positive bacteria include the causative agents of the diseases diphtheria, anthrax, tetanus, scarlet fever, and certain forms of pneumonia and tonsillitis. Gram-negative bacteria include organisms that cause typhoid fever, dysentery, gonorrhea and whooping cough.

Since the Gram stain results reflect differences in cell wall structure, the classification of a bacterium as Gram positive or Gram negative has significant clinical implications. As a general rule, the presence of the lipid-containing outer capsule of Gram negative bacteria often is associated with increased virulence and pathogenicity. Additionally, Gram-negative bacteria have lipopolysaccharide in their outer membrane, an endotoxin which increases the severity of inflammation. This inflammation may be so severe that septic shock may occur. Gram-positive infections are generally less severe because the human body does not contain peptidoglycan, and thus the cell wall can be readily targeted by antibiotics (e.g., penicillin) or host enzymes (such as lysozyme in tears). Certain Gram-positive bacteria (e.g., Mycobacterium tuberculosis and other agents of tuberculosis, or Nocardia species, the agents of nocardiosis are, however, quite virulent.

The Gram stain is positive in approximately 70% of patients with acute bacterial meningitis. A negative Gram stain and/or bacterial culture does however not exclude infection, particularly when the patient has received antibiotics. If an anaerobic organism is suspected, special cultures are presently required. Cultures may take four weeks to become positive (Bhistikul, D. et al (1994) “The Role of Bacterial Antigen Detection Tests In The Diagnosis Of Bacterial Meningitis,” Ped. Emer. Care 10(2):67-71; Negrini, B. et al. (2000) “Cerebrospinal Fluid Findings In Aspetic Versus Bacterial Meningitis,” Pediatrics 105(2):316-319; Ray, P. et al. (2007) “Accuracy Of The Cerebrospinal Fluid Results To Differentiate Bacterial From Non-Bacterial Meningitis, In The Case Of Negative Gram-Stained Smear,” Amer. J. Emerg. Med. 25:179-184).

The detection of bacterial antigens of Neisseria meningitidis, Haemophilus influenzae type b, Streptococcus pneumoniae or, in infants, Group B streptococcus, have been proposed as useful for the diagnosis of meningitis (Sormunen, P. et al. (1999) “C-Reactive Protein Is Useful In Distinguishing Gram-Stain Negative Bacterial Meningitis From Viral Meningitis In Children,” J. Pediatrics 134(6):162-171; Dubos, F. et al (2006) “Serum Procalcitonin And Other Biologic Markers To Distinguish Between Bacterial and Aseptic Meningitis”. Pediatrics 149:72-76; Lorino, G. et al. (2000) “Diagnostic Values Of Cytokine Assays In Cerebrospinal Fluid In Culture-Negative, Polymerase Chain Reaction-Positive Bacterial Meningitis,” Eur. J. Clin. Microbiol. Infect. Dis. 19:388-392; Murakami, S. et al. (epub Mar. 19, 2008) “Diagnosis Of Tuberculous Meningitis Due To ESAT-6-Specific IFN-γ Production Detected By Enzyme-Linked Immunospot Assay In Cerebrospinal Fluid,” Clin. Vaccine Immunol.; Inada, K. et al. (2003) “A Silkworm Larvae Plasma Test For Detecting Peptidoglycan In Cerebrospinal Fluid Is Useful For The Diagnosis of Bacterial Meningitis,” Microbiol. Immunol. 47(10):701-707; Dyson, D. et al. (1976) “Use of Limulus Lysate For Detecting Gram-Negative Neonatal Meningitis” Pediatrics 58(1):105-109; Ross, S. et al. (1975) “Limulus Lysate Test For Gram-Negative Bacterial Meningitis. Bedside Application,” J. Amer. Med. Assn. 233(13):1366-1369).

High protein levels are found in conditions such as meningeal inflammation (e.g., purulent or tuberculous meningitis) or with increased vascular (blood-brain) permeability (e.g., viral meningitis (Sormunen, P. et al. (1999) “C-Reactive Protein Is Useful In Distinguishing Gram-Stain Negative Bacterial Meningitis From Viral Meningitis In Children,” J. Pediatrics 134(6):162-171; Negrini, B. et al. (2000) “Cerebrospinal Fluid Findings In Aseptic Versus Bacterial Meningitis,” Pediatrics 105(2):316-319; Murakami, S. et al. (epub. Mar. 19, 2008) “Diagnosis Of Tuberculous Meningitis Due To ESAT-6-Specific IFN-γ Production Detected By Enzyme-Linked Immunospot Assay In Cerebrospinal Fluid,” Clin. Vaccine Immunol.; Jorgensen, J. H. et al (1978) “Rapid Diagnosis Of Gram-Negative Bacterial Meningitis By The Limulus Endotoxin Assay,” J. Clin. Micro. 7(1):12-17; Chavanet et al. (2007) “Performance Of A Predictive Rule To Distinguish Bacterial and Viral Meningitis,” J. Infection 54:328-336).

Unfortunately, diagnostic test results for meningitis can take up to a week to obtain. The delay in obtaining confirmation of the disease can be a severe problem in light of the often life-threatening nature of bacterial meningitis. Thus, despite all prior advances in pathogen diagnosis, a need remains for a rapid assay capable of rapidly determining whether a biological fluid contains a suspect Gram positive bacterial, a Gram negative bacterial or a viral pathogen. The present invention is directed to this and other needs.

### SUMMARY OF THE INVENTION

The present invention relates to devices and methods for rapidly determining whether a biological fluid contains a suspect Gram positive bacterial, a Gram negative bacterial or a viral pathogen. The invention particularly pertains to such devices and methods wherein the biological fluid is cerebrospinal fluid, and wherein the suspect pathogen is a causative agent of meningitis.

In detail, the invention provides a dip-stick device suitable for simultaneous immunochromatographic analysis of two or more assessed analytes potentially contained in a fluid sample, wherein the device comprises a solid support possessing three or more planar longitudinal faces, and at least one first and one second porous carrier affixed to at least one face thereof, the first and second porous carriers of each face being in fluid contact with one another, but spatially distinct from each other; wherein for each longitudinal face of the device having affixed carriers, the first porous carrier comprises a detectably labeled detector molecule capable of binding to one of the assessed analytes and the second such porous carrier contains an immobilized, but unlabeled capture molecule capable of binding to the assessed analyte.

The invention further concerns the embodiment of the above-described dip-stick device wherein the device has three planar longitudinal faces, each such face having affixed thereto one of the first and second porous carriers, wherein the first porous carrier affixed to a first such face comprises a detectably labeled detector molecule capable of binding to an assessed analyte whose presence is characteristic of a Gram positive bacteria;

wherein the first porous carrier affixed to a second such face comprises a detectably labeled detector molecule capable of binding to an assessed analyte whose presence is characteristic of a Gram negative bacteria;
wherein the first porous carrier affixed to a third such face comprises a detectably labeled detector molecule capable of binding to an assessed analyte whose presence is characteristic of a viral pathogen.

The invention further concerns the embodiments of the above-described dip-stick device wherein the detectably labeled detector molecule capable of binding to an assessed analyte characteristic of a Gram positive bacteria is melanin.

The invention further concerns the embodiments of the above-described dip-stick device wherein the detectably labeled detector molecule capable of binding to an assessed analyte characteristic of a Gram negative bacteria is boc-Leu-Gly-Arg-paranitroaniline.

The invention further concerns the embodiments of the above-described dip-stick device wherein the detectably labeled detector molecule capable of binding to an assessed analyte characteristic of a viral pathogen is an antibody that immunospecifically reacts with a non-polio enterovirus, a paramyxovirus, an arbovirus, a herpes virus, a lymphocytic choriomeningitis virus, an adenovirus, a measles virus, or a human immunodeficiency virus.

The invention further concerns the embodiments of the above-described dip-stick device wherein the detectable label is an enzyme, and the dip-stick device additionally comprises a chromogenic substrate for such enzyme.

The invention further concerns a method for conducting the simultaneous immunochromatographic analysis of two or more assessed analytes potentially contained in a fluid sample, wherein the method comprises the steps:
(A) Incubating a biological fluid in the presence of a device that comprises a solid support possessing three or more planar longitudinal faces, and at least one first and one second porous carrier affixed to at least one face thereof, the first and second porous carriers of each face being in fluid contact with one another, but spatially distinct from each other; wherein for each longitudinal face of the device having affixed carriers, the first porous carrier comprises a detectably labeled detector molecule capable of binding to one of the analytes to be assessed and the second such porous carrier contains an immobilized, but unlabeled capture molecule capable of binding to the assessed analyte;
(B) Permitting molecules of assessed analyte, if present, to migrate into the first porous carrier and to react with, or bind to, detectably labeled detector molecule present therein;
(C) Permitting molecules of assessed analyte, if present, that have reacted with, or bound to, the detectably labeled detector molecule to migrate into the second porous carrier and become immobilized to the capture molecule;
(D) Detecting whether detectably labeled detector molecules are immobilized to the immobilized capture molecules of the second porous carrier;
   wherein detection of detectably labeled detector molecules immobilized to the immobilized capture molecules of the second porous carrier is indicative of the presence of the assessed molecule in the biological fluid.

The invention further concerns the embodiment of such method wherein the device has three planar longitudinal faces, each such face having affixed thereto one of the first and second porous carriers, wherein the first porous carrier affixed to a first such face comprises a detectably labeled detector molecule capable of binding to an assessed analyte whose presence is characteristic of a Gram positive bacteria;

wherein the first porous carrier affixed to a second such face comprises a detectably labeled detector molecule capable of binding to an assessed analyte whose presence is characteristic of a Gram negative bacteria;
wherein the first porous carrier affixed to a third such face comprises a detectably labeled detector molecule capable of binding to an assessed analyte whose presence is characteristic of a viral pathogen.

The invention further concerns the embodiment of such method wherein the detectably labeled detector molecule capable of binding to an assessed analyte characteristic of a Gram positive bacteria is melanin.

The invention further concerns the embodiment of such method wherein the detectably labeled detector molecule capable of binding to an assessed analyte characteristic of a Gram negative bacteria is boc-Leu-Gly-Arg-paranitroaniline.

The invention further concerns the embodiment of such method wherein the detectably labeled detector molecule is capable of binding to an assessed analyte characteristic of a viral pathogen is an antibody that immunospecifically reacts with a non-polio enterovirus, a paramyxovirus, an arbovirus, a herpes virus, a lymphocytic choriomeningitis virus, an adenovirus, a measles virus, or a human immunodeficiency virus.

The invention further concerns the embodiment of such method wherein the detectable label is an enzyme, and the dip-stick device additionally comprises a chromogenic substrate for such enzyme.

### BRIEF DESCRIPTION OF THE DRAWINGS

FIG. 1 shows an example of a preferred triangular prism device for analyzing biological fluids.

FIG. 2 shows a preferred device of the present invention in use.

FIG. 3 illustrates results obtainable through the use of the preferred devices of the present invention.

### DETAILED DESCRIPTION OF THE INVENTION

The present invention relates to devices and methods for rapidly determining whether a biological fluid contains a suspect Gram positive bacterial, a Gram negative bacterial or a viral pathogen. The invention particularly pertains to such devices and methods wherein the biological fluid is cerebrospinal fluid, and wherein the suspect pathogen is a causative agent of meningitis.

Any of a variety of biological fluids are amenable to analysis using the devices and methods of the present invention. Such fluids include cerebrospinal fluid, synovial fluid, blood, serum, plasma, saliva, intestinal fluids, semen, tears, nasal secretions, etc. It will be appreciated that any fluidic biological sample (e.g., tissue or biopsy extracts, extracts of feces, sputum, etc.) may likewise be employed in accordance with the present invention. The invention is particularly concerned with the analysis of pathogens potentially present in cerebrospinal fluid, and particularly those responsible for meningitis. Cerebrospinal fluid (“CSF”) is a bodily fluid that occupies the subarachnoid space and the ventricular system around and inside the brain and the spinal cord. CSF is produced in the brain by modified ependymal cells in the choroid plexus. It circulates from the choroid plexus through the interventricular foramina (foramen of Monro) into the third ventricle, and then through the mesencephalic duct (cerebral aqueduct) into the fourth ventricle, where it exits through two lateral apertures (foramina of Luschka) and one median aperture (foramen of Magendie). It then flows through the cerebromedullary cistern down the spinal cord and over the cerebral hemispheres. CSF is produced at a rate of 500 ml/day. Approximately 135-150 ml are retained in the brain, with the remainder draining into the circulatory system. CSF is generally sampled by inserting a spinal needle between the lumbar vertebrae L3/L4 or L4/L5 to a depth sufficient to permit collection of the fluid, a procedure known as Lumbar puncture (Farley, A. et al. (2008) “Lumbar Puncture,” Nurs. Stand. 22(22):46-48; Riou, E. M. et al. (2008) “Cerebrospinal Fluid Analysis In The Diagnosis And Treatment Of Arterial Ischemic Stroke,” Pediatr. Neurol. 38(1):1-9; Sempere, A. P. et al. (2007) “Lumbar Puncture: Its Indications, Contraindications, Complications And Technique,” Rev. Neurol. 45(7):433-436).

In healthy individuals, CSF is a clear and non-viscous fluid. A finding that CSF is yellow, turbid, or viscous is indicative of bacterial infection. A yellowish turbid fluid is suggestive of pyogenic bacterial meningitis. A yellowish viscous fluid is suggestive of tuberculous meningitis or fungal meningitis. Significantly, however, disease can be present even if the CSF is clear since viral meningitis does not cause a clouding or coloring of the fluid.

The present invention accomplishes the rapid analysis and simultaneous analysis of properties of pathogens that may be present in a sampled biological fluid. Such properties may include Gram positiveness, Gram negativeness, the presence of a viral antigen, the presence of a bacterial antigen, the presence of a fungal (including yeast) antigen, the presence of a host antibody elicited by a viral antigen, the presence of a host antibody elicited by a bacterial antigen, the presence of a host antibody elicited by the presence of fungal (including yeast) antigen, etc. As used herein, the term “rapid” refers to an analysis that provides an assessment of a property within 2 hours, more preferably within 1 hour, more preferably still within 30 minutes, 15 minutes, 10 minutes, 5 minutes, 1 minute or most preferably, less than one minute.

### A. The Preferred Method of Analysis of the Devices of the Present Invention

The present invention employs an immunoassay to determine whether a molecule that is characteristic of a predetermined property is present in the sampled biological fluid. Any of a wide variety of assay formats may be used in accordance with the methods of the present invention. Such formats may be heterogeneous or homogeneous, sequential or simultaneous, competitive or noncompetitive. U.S. Pat. Nos. 5,563,036; 5,627,080; 5,633,141; 5,679,525; 5,691,147; 5,698,411; 5,747,352; 5,811,526; 5,851,778; and 5,976,822 illustrate several different assay formats and applications. Such assays can be formatted to be quantitative, so as to measure the concentration, amount or user defined upper or lower limits of concentration or amount of an antibody or antigen, or they may be formatted to be qualitative, to measure the presence or absence of an antibody or antigen.

Heterogeneous immunoassay techniques typically involve the use of a solid phase material to which the reaction product becomes bound, but may be adapted to involve the binding of non-immobilized antigens and antibodies (i.e., a solution-phase immunoassay). The reaction product is separated from excess sample, assay reagents, and other substances by removing the solid phase from the reaction mixture (e.g., by washing). One type of solid phase immunoassay that may be used in accordance with the present invention is a sandwich immunoassay. In the sandwich assay, the more analyte present in the sample, the greater the amount of label present on the solid phase. This type of assay format is generally preferred, especially for the visualization of low analyte concentrations, because the appearance of label on the solid phase is more readily detected.

In accordance with one embodiment of the present invention, a “capture” molecule that is immunospecifically reactive with a molecule that is characteristic of the predetermined property being assessed (i.e., the “assessed analyte”) is immobilized to a solid support and incubated in contact with the biological fluid being sampled. The capture molecule may be an antibody or other binding molecule. As will be appreciated, such capture molecule may be alternatively be incubated with the biological sample in an unbound state and then subsequently bound to the solid support (i.e., it may immobilizable). The supports are then preferably extensively treated (e.g., by washing, etc.) to substantially remove molecules of the biological fluid that failed to bind to the immobilized/immobilizable capture molecule. In consequence of such treatment, if the biological fluid contains the assessed analyte, such molecule will be immobilized to the solid support.

A detectably labeled antibody (e.g., an anti-human IgG antibody or an antibody capable of binding to the assessed analyte) is then preferably added and the support is incubated under conditions sufficient to permit the antibody to bind to any of the assessed analyte that has been immobilized to the support. The support is then preferably extensively treated (e.g., by washing, etc.) to substantially remove any unbound detectably labeled antibody. If the assessed analyte is present in the tested sample, an immobilized, and detectable immune complex will form (i.e., detectably labeled antibody/assessed analyte/capture molecule). In such an assay, the detection of detectably labeled antibody bound to the support is indicative of the presence of the assessed analyte in the fluid being tested. Sandwich assay formats are described by Schuurs et al. U.S. Pat. Nos. 3,791,932 and 4,016,043, and by Pankratz, et al., U.S. Pat. No. 5,876,935. The detectably labeled antibody may be a natural immunoglobulin isolated from nonhuman primates (e.g., anti-human IgG murine antibody, anti-human IgG goat antibody, etc.), or can be produced recombinantly or synthetically. It may be an intact immunoglobulin, or an immunoglobulin fragment (e.g., FAb, F(Ab)₂, etc.). As desired, other binding molecules (capable of binding to the assessed analyte) may be employed in concert with or in lieu of such antibodies. For example, the assessed analyte can be biotinylated and the antibody can be replaced with labeled avidin or streptavidin.

To eliminate the need to separate bound from free reagents and reduce the time and equipment needed for the assay, a homogeneous assay format may alternatively be employed. In such assays, one component of the binding pair may still be immobilized; however, the presence of the second component of the binding pair is detected without a bound-free separation. Examples of homogeneous optical methods are the EMIT method of Syva, Inc. (Sunnyvale, Calif.), which operates through detection of fluorescence quenching; the laser nephelometry latex particle agglutination method of Behringwerke (Marburg, Germany), which operates by detecting changes in light scatter; the LPIA latex particle agglutination method of Mitsubishi Chemical Industries (Tokyo, Japan); the TDX fluorescence depolarization method of Abbott Laboratories (Abbott Park, Ill.); and the fluorescence energy transfer method of Cis Bio International (Paris, France). Any of such assays may be adapted for use in accordance with the objectives of the present invention.

The binding assay of the present invention may be configured as a competitive assay. In a competitive assay, the more assessed analyte present in the test sample, the lower the amount of label immobilized onto the solid support. In a manner similar to the sandwich assay, the competitive assay can be conducted by providing a defined amount of a labeled assessed analyte and determining whether the fluid being tested contains such molecule by its ability to compete with the labeled antibody for binding to the support. In such a competitive assay, the amount of captured labeled antibody is inversely proportional to the amount of analyte present in the test sample. Smith (U.S. Pat. No. 4,401,764) describes an alternative competitive assay format using a mixed binding complex that can bind analyte or labeled analyte but in which the analyte and labeled analyte cannot simultaneously bind the complex. Clagett (U.S. Pat. No. 4,746,631) describes an immunoassay method using a reaction chamber in which an analyte/ligand/marker conjugate is displaced from the reaction surface in the presence of test sample analyte and in which the displaced analyte/ligand/marker conjugate is immobilized at a second reaction site. The conjugate includes biotin, bovine serum albumin, and synthetic peptides as the ligand component of the conjugate, and enzymes, chemiluminescent materials, enzyme inhibitors, and radionucleotides as the marker component of the conjugate. Li (U.S. Pat. No. 4,661,444) describes a competitive immunoassay using a conjugate of an anti-idiotype antibody and a second antibody, specific for a detectable label, in which the detectable response is inversely related to the presence of analyte in the sample. Allen (European Patent Appln. No. 177,191) describes a binding assay involving a conjugate of a ligand analog and a second reagent, such as fluorescein, in which the conjugate competes with the analyte (ligand) in binding to a labeled binding partner specific for the ligand, and in which the resultant labeled conjugate is then separated from the reaction mixture by means of solid phase carrying a binding partner for the second reagent. This binding assay format combines the use of a competitive binding technique and a reverse sandwich assay configuration; i.e., the binding of conjugate to the labeled binding member prior to separating conjugate from the mixture by the binding of the conjugate to the solid phase. The assay result, however, is determined as in a conventional competitive assay in which the amount of label bound to the solid phase is inversely proportional to the amount of analyte in the test sample. Chieregatt et al. (GB Patent No. 2,084,317) describe a similar assay format using an indirectly labeled binding partner specific for the analyte. Mochida et al. (U.S. Pat. No. 4,185,084) also describe the use of a double-antigen conjugate that competes with an antigen analyte for binding to an immobilized antibody and that is then labeled. This method also results in the detection of label on a solid phase in which the amount of label is inversely proportional to the amount of analyte in the test sample. Sadeh et al. (U.S. Pat. No. 4,243,749) describe a similar enzyme immunoassay in which a hapten conjugate competes with analyte for binding to an antibody immobilized on a solid phase. Any of such variant assays may be used in accordance with the present invention.

In all such assay formats, at least one component of the assay reagents will preferably be labeled or otherwise detectable, most preferably by a label that causes the evolution or quenching of light. Such component may be an antibody, the assessed analyte, or a molecule that binds the assessed analyte, depending on the immunoassay format employed. Radioisotopic-binding assay formats (e.g., a radioimmunoassay, etc.) employ a radioisotope as such label; the signal is detectable by the evolution of light in the presence of a fluorescent or fluorogenic moiety (see, U.S. Pat. Nos. 5,698,411; 5,976,822). Enzymatic-binding assay formats (e.g., an ELISA, etc.) employ an enzyme as a label; the signal is detectable by the evolution of color or light in the presence of a chromogenic or fluorogenic moiety. Other labels, such as paramagnetic labels, materials used as colored particles, latex particles, colloidal metals such as selenium and gold, and dye particles (see U.S. Pat. Nos. 4,313,734; 4,373,932, and 5,501,985) may also be employed. The use of enzymes (especially alkaline phosphatase, β-galactosidase, horse radish peroxidase, or urease) as the detectable label (i.e., an enzyme immunoassay or EIA) is preferred. The presence of enzymatic labels may be detected through the use of chromogenic substrates (including those that evolve or adsorb fluorescent, UV, visible light, etc.) in response to catalysis by the enzyme label. Alternatively, chemical labels may be employed (e.g., colloidal gold, latex bead labels, etc.). Detection of label can be accomplished using multiple detectors, multipass filters, gratings, or spectrally distinct fluors (see e.g., U.S. Pat. No. 5,759,781), etc. For example, peroxidase may be employed as an enzyme label in concert with the chromogenic substrate 3, 3′, 5,5′-tetramethylbenzidine (TMB). The periodate technique may be used to label molecules with peroxidase (Nakane, P. K. et al. (1974) “Peroxidase-Labeled Antibody. A New Method Of Conjugation,” J. Histochem. Cytochem. 22:1084-1090) or the partners may be linked with a heterobifunctional reagent (Ishikawa, E. et al. (1983) “Enzyme-Labeling Of Antibodies And Their Fragments For Enzyme Immunoassay And Immunohistochemical Staining,” J. Immunoassay 4(3):209-327).

Any of a wide variety of solid supports may be employed in the immunoassays of the present invention. Suitable materials for the solid support are synthetics such as polystyrene, polyvinyl chloride, polyamide, or other synthetic polymers, natural polymers such as cellulose, as well as derivatized natural polymers such as cellulose acetate or nitrocellulose, and glass, especially glass fibers.

The present invention particularly relates to the use of an immunochromatographic assay format to detect the assessed analytes. In a preferred immunochromatographic assay format, two contacting, but spatially distinct, porous carriers are employed. The first such carrier will contain a non-immobilized, detectably labeled detector molecule capable of binding to the assessed analyte and the second such carrier will contain an immobilized, but unlabeled capture molecule capable of binding to the assessed analyte. In accordance with the principles of an immunochromatographic assay, the first carrier is placed in contact with the biological fluid. Capillary action or suction, etc. causes molecules of the fluid to migrate into the first carrier. If the fluid contains the assessed analyte, such molecule will become bound to the detector molecule. Since neither the assessed analyte nor the detector molecule have been immobilized to the support, both molecules continue to migrate (but as a complex) into the first carrier. Migration of the complex will continue until the complex enters the second carrier. There, immobilized capture molecules will bind to the complex and immobilize it to the support. The presence of detector molecule bound to the support in the region of the second carrier is indicative of the presence of assessed analyte in the sample. If desired a control antibody or binding molecule may be immobilized to a third porous carrier, contacting, but spatially distinct, from the second carrier. Molecules entering the third carrier become immobilized to such control molecules and are then detected as evidence that the test reagents are functioning properly. The assay can be made quantitative by measuring the quantity of detector molecule that becomes bound within the second porous carrier.

For analyzing whether a sampled fluid contains Gram positive bacteria or fungi, the present invention particularly relates to the use of an immunochromatographic assay using the silkworm larvae plasma test for detecting the presence of peptidoglycan (Inada, K. et al. (2003) “A Silkworm Larvae Plasma Test For Detecting Peptidoglycan In Cerebrospinal Fluid Is Useful For The Diagnosis of Bacterial Meningitis,” Microbiol. Immunol. 47(10):701-707; Shimizu, T. et al. (2005) “Diagnostic And Predictive Value Of The Silkworm Larvae Plasma Test For Postoperative Infection Following Gastrointestinal Surgery,” Crit. Care Med. 33(6):1288-1295; Kobayashi, T. et al. (2000) “Detection Of Peptidoglycan In Human Plasma Using The Silkworm Larvae Plasma Test,” FEMS Immunol Med. Microbiol. 28(1):49-53; Hiyoshi, M. et al. (1999) “A Clinical Evaluation Of The New Laboratory Method That Diagnoses Bacterial Infection, Using Silkworm Larvae Plasma,” Kansenshogaku Zasshi. 73(12):1222-1226; Tsuchiya, M. et al. (1996) “Detection Of Peptidoglycan And Beta-Glucan With Silkworm Larvae Plasma Test,” FEMS Immunol Med Microbiol. 15(2-3):129-134). In this test, the prophenol-oxidase reaction that occurs when silkworm larvae plasma interacts with peptidoglycan (an essential component of gram-positive bacterial cell walls and fungi) results in the formation of melanin. Therefore, in a preferred embodiment, the first carrier of the invention will contain a labeled, non-immobilized antibody to melanin. In accordance with the principles of the invention, the labeled, non-immobilized anti-melanin antibody is placed in contact with the biological sample, preferably cerebrospinal fluid. Capillary action or suction, etc. causes molecules of the biological sample to migrate into contact with said antibody. If the biological sample contains gram-positive bacteria or fungi, the silkworm larvae plasma will react with the peptidoglycan in their cell walls, resulting in the production of melanin. The produced melanin will become bound to the labeled, non-immobilized anti-melanin antibody present in the first porous carrier. Since neither the melanin nor the labeled anti-melanin antibody have been immobilized to the support, both molecules will continue to migrate (but as a complex) through the first porous carrier of the immunochromatographic assay. Migration of the complex will continue until the complex enters the second portion of the immunochromatographic assay. There, immobilized capture molecules such as anti-IgG antibodies or Protein A or G will bind the complex and immobilize it to the support. The presence of detector molecule bound to the support in the second region of the assay is indicative of the presence of melanin in the sample, and thus, the presence of peptidoglycan, which is indicative of a gram positive or fungal infection of the cerebrospinal fluid. If desired a control antibody or binding molecule may be immobilized to a third porous carrier, contacting, but spatially distinct, from the second carrier. Molecules entering the third carrier become immobilized to such control molecules and are then detected as evidence that the test reagents are functioning properly. The assay can be made quantitative by measuring the quantity of detector molecule that becomes bound within the second porous carrier.

For analyzing whether a sampled fluid contains Gram negative bacteria, the present invention particularly relates to the use of an immunochromatographic assay using the limulus lysate test for detecting the presence of lipopolysaccharide (Nachum, R. et al. (1973) “Rapid Detection Of Gram-Negative Bacterial Meningitis By The Limulus Lysate Test,” N. Engl. J. Med. 289(18):931-934; Dyson, D. et al. (1976) “Use of Limulus Lysate For Detecting Gram-Negative Neonatal Meningitis” Pediatrics 58(1):105-109; Ross, S. et al. (1975) “Limulus Lysate Test For Gram-Negative Bacterial Meningitis. Bedside Application,” J. Amer. Med. Assn. 233(13):1366-1369)). In this test, the LPS endotoxin of Gram negative bacteria activates Factor C in the Limulus lysate and thereby initiates a cascade that leads to coagulation and coagulin production. The assay can be made calorimetric by the addition of the synthetic chromogenic substrate, boc-Leu-Gly-Arg-paranitroaniline. The release of the chromagen is detected with diazo-coupling agents and is measured photometrically at 545 nm. In a preferred embodiment, the first carrier of the invention will be a labeled, non-immobilized antibody to coagulin. In accordance with the principles of the invention, the labeled, non-immobilized anti-coagulin antibody is placed in contact with the biological sample, preferably cerebrospinal fluid. Capillary action or suction, etc. causes molecules of the biological sample to migrate into contact with the antibody. If the biological sample contains gram-negative bacteria, their LPS endotoxin will react with the Limulus lysate, resulting in the production of coagulin. The coagulin will become bound to the labeled, non-immobilized anti-coagulin antibody present in the first porous carrier. Since neither the coagulin nor the labeled anti-coagulin antibody have been immobilized to the support, both molecules will continue to migrate (but as a complex) through the first porous carrier of the immunochromatographic assay. Migration of the complex will continue until the complex enters the second portion of the immunochromatographic assay. There, immobilized capture molecules such as anti-IgG antibodies or Protein A or G will bind the complex and immobilize it to the support. The presence of detector molecule bound to the support in the second region of the assay is indicative of the presence of coagulin in the sample, and thus, the presence of Gram negative bacteria in the sampled cerebrospinal fluid. If desired a control antibody or binding molecule may be immobilized to a third porous carrier, contacting, but spatially distinct, from the second carrier. Molecules entering the third carrier become immobilized to such control molecules and are then detected as evidence that the test reagents are functioning properly. The assay can be made quantitative by measuring the quantity of detector molecule that becomes bound within the second porous carrier.

For analyzing whether a sampled fluid contains a viral pathogen, the present invention particularly relates to the use of an immunochromatographic assay using antibody directed against antibodies that have been elicited by the virus. Therefore, in a preferred embodiment, the first carrier of the invention will contain a labeled, non-immobilized viral antigen or a labeled, non-immobilized antibody capable of immunospecifically binding to antibodies that have been elicited by the virus. In accordance with the principles of the invention, the labeled, non-immobilized viral antigen or a labeled, non-immobilized antibody capable of immunospecifically binding to antibodies that have been elicited by the virus is placed in contact with the biological sample, preferably cerebrospinal fluid. Capillary action or suction, etc. causes molecules of the biological sample to migrate into contact with said antibody. If the biological sample contains antibodies that have been elicited by the virus they will become bound to the labeled, non-immobilized viral antigen or labeled, non-immobilized antibody present in the first porous carrier. Since the molecules have been immobilized to the support, antibodies that have been elicited by the virus if present that have become bound to the labeled, non-immobilized viral antigen or labeled, non-immobilized antibody will continue to migrate (but as a complex) through the first porous carrier of the immunochromatographic assay. Migration of the complex will continue until the complex enters the second portion of the immunochromatographic assay. There, immobilized capture molecules such as anti-IgG antibodies or Protein A or G will bind the complex and immobilize it to the support. The presence of detector molecule bound to the support in the second region of the assay is indicative of the presence of antibodies that have been elicited by the virus, and thus, the presence of viral pathogens in the cerebrospinal fluid. If desired a control antibody or binding molecule may be immobilized to a third porous carrier, contacting, but spatially distinct, from the second carrier. Molecules entering the third carrier become immobilized to such control molecules and are then detected as evidence that the test reagents are functioning properly. The assay can be made quantitative by measuring the quantity of detector molecule that becomes bound within the second porous carrier.

### B. The Preferred Devices of the Present Invention

In preferred embodiments, the invention employs a dip-stick device suitable for immunochromatographic analysis comprising the above-mentioned porous carriers. Preferably, the device will additionally comprise a casing constructed of, for example, a plastic material, paper, etc., to which the porous carriers are affixed. The casing is preferably formed in the shape of a prism (and preferably a right prism) composed of a plurality of planar longitudinal faces and a top and bottom end faces and serves to provide structural integrity to the dip-stick. The porous carriers are preferably rectangular and affixed to planar longitudinal faces. Alternatively, the porous chromatographic supports of the device may be fashioned to possess sufficient rigidity and structure that they may be directly combined to form the prism (without any requirement for an underlying inert support).

By affixing the above-described porous carriers to at least one and preferably all of the longitudinal faces of the device, such faces acquire the capability of analyzing more than two properties, more preferably only two properties, or most preferably only a single property that may potentially be possessed by a pathogen or by the biological fluid in response to pathogen presence therein, as determined by the substituents of the porous carriers. The end faces of the devices of the present invention may be closed or open (i.e., absent), so as to create a prism that is either solid or hollow. Preferably the end faces of the device will be absent and the prism will be hollow. In a preferred embodiment, the prism will be prepared by folding or cutting a planar support material so as to form supports for the longitudinal faces of the prism. As will be appreciated, the prismatic nature of the device greatly enhances the structural strength of the individual porous chromatographic supports.

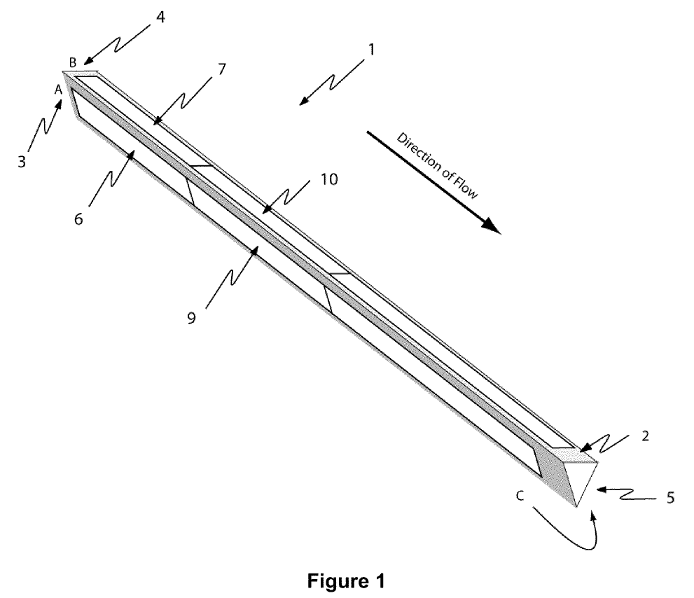

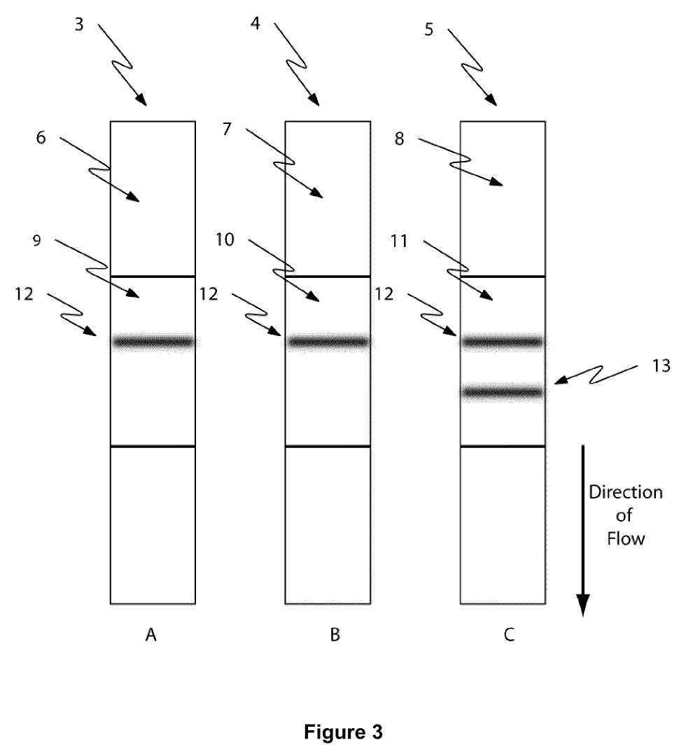

In a preferred embodiment, the device 1 is a triangular prism as shown in FIG. 1. It will be appreciated that device 1 is not shown to scale. The porous carriers affixed to the support 2 that forms the respective three longitudinal faces of the prism are designed to permit simultaneous analysis of three properties. Most preferably such three properties are whether the biological fluid contains: (1) Gram positive bacteria, (2) Gram negative bacteria, or (3) a viral pathogen. As shown in FIG. 1, device 1 is formed from a triangular prism support 2 having porous carriers A, B and C (3, 4 and 5, respectively). Porous carriers A and B are visibly affixed to front longitudinal faces of the prism, with porous carrier C 5 affixed to the (hidden from view) rear face of the prism. First porous carrier regions 6 and 7 of porous carriers A and B, respectively (and a corresponding region 8 of porous carrier C (shown in FIG. 3, not shown in FIG. 1) comprise an absorbent region that contains labeled detector molecule specific for binding to different assessed analytes (such that the device is capable of detecting the potential presence of three different assessed analytes). Second porous carrier regions 9 and 10 of porous carriers A and B, respectively (and a corresponding region 11 of porous carrier C (shown in FIG. 3, not shown in FIG. 1) contain immobilized capture molecule specific for binding to the different assessed analytes and comprise the “reading zone” for determining the outcome of the assay. A preferred direction of analyte flow is indicated; flow can go in the opposite direction is desired.

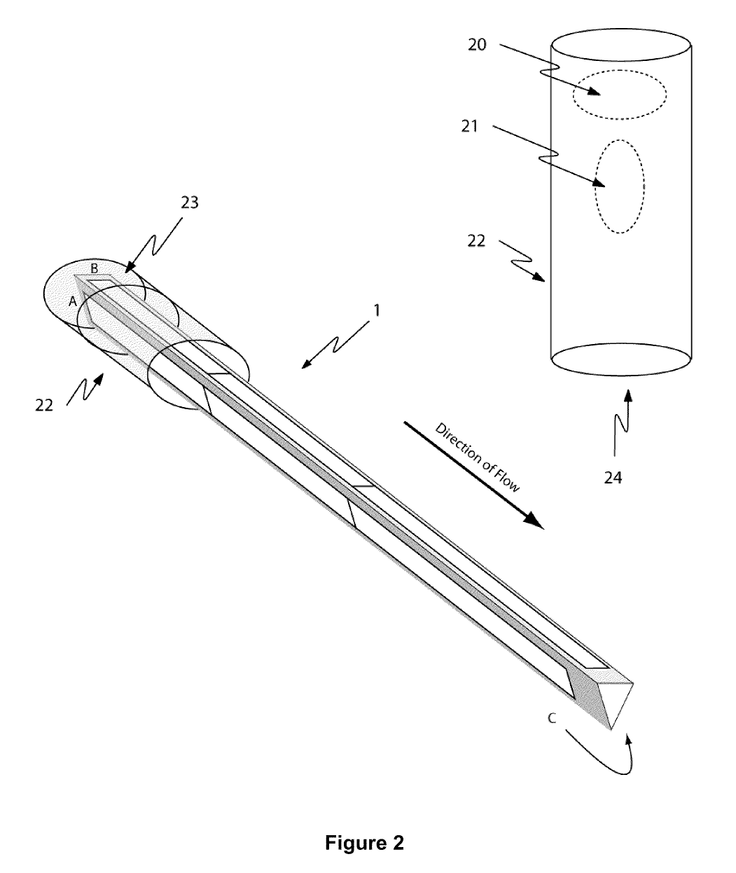

FIG. 2 shows a preferred device of the present invention in use. Biological fluid is introduced into a chamber 20 of an end-cap receptacle 22 for the device (having a receiving opening 24). End-cap receptacle 22 preferably tightly mates in a snug, snap-fitting with device 1. Buffer, labeled molecules, etc., are contained in a second end-cap chamber 21. Introduction of the dip-stick device 1 into the end-cap receptacle 22 pierces chambers 20 and 21 and allows their contents to mix to form reservoir 23. Reservoir 23 is in contact with porous carriers A, B, and C (3, 4 and 5, respectively) and such contact permits the analytes of reservoir 23 to migrate along the porous carriers 3, 4 and 5.

FIG. 3 illustrates the results obtained through the use of the present invention. Panel A 3 corresponds to porous carriers designed to identify Gram negative pathogens. The absence of detectable label 13 in reading zone 9 indicates that the fluid being tested does not contain Gram negative bacteria; the presence of a control detectable signal 12 indicates that the reagents are functioning properly. Panel B 4 corresponds to porous carriers designed to identify Gram positive pathogens. The absence of detectable label 13 in reading zone 10 indicates that the fluid being tested does not contain Gram negative bacteria; the presence of a control detectable signal 12 indicates that the reagents are functioning properly. Panel C 5 corresponds to porous carriers designed to identify viral pathogens. The presence of detectable label 13 indicates that the fluid being tested contains viral pathogens; the presence of a control detectable signal 12 indicates that the reagents are functioning properly. In a preferred embodiment, dyes or chromogenic reagents are employed so that the assay results can be readily scored by color (e.g., a positive result for a test of Gram positiveness would display with a dark blue-violet color; a positive result for a test of Gram negativeness would display with a red-pink color; a positive result for a test of virus-specific antibody would display with a yellow color, etc.).

Materials for use in the assay of the invention are ideally suited for the preparation of a kit. Such a kit may comprise a carrier means being compartmentalized to receive in close confinement; one or more containers means vials, tubes and the like; each of the containers means comprising one of the separate elements to be used in the method. For example, one of the containers means may comprise a suitable binding molecule bound to a solid support. A second container may comprise soluble, detectably labeled antibody, preferably in lyophilized form, or in solution. In addition, the kit may also contain one or more containers, each of which comprises a (different) predetermined amount of control reagents. These latter containers can be used to prepare a standard curve into which can be interpolated the results obtained from the sample containing the unknown amount of assessed analytes.

In using the kit, all the user has to do is add to a container a premeasured amount of a sample suspected of containing a measurable yet unknown amount of assessed analyte, a premeasured amount of support-bound antigen present in the first container, and a premeasured amount of the detectably labeled antibody present in the second container. After an appropriate time for incubation, an immune complex is formed and is separated from the supernatant fluid, and the immune complex or the supernatant fluid are detected, as by radioactive counting, addition of an enzyme substrate, and color development, or by inclusion of a chemical label (e.g., colloidal gold, latex beads, etc.).

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A dip-stick device suitable for simultaneous immunochromatographic analysis of two or more assessed analytes potentially contained in a fluid sample, wherein said device comprises a solid support possessing three or more planar longitudinal faces, and at least one first and one second porous carrier affixed to at least one face thereof, said first and second porous carriers of each face being in fluid contact with one another, but spatially distinct from each other; wherein for each longitudinal face of said device having affixed carriers, said first porous carrier comprises a detectably labeled detector molecule capable of binding to one of said assessed analytes and the second such porous carrier contains an immobilized, but unlabeled capture molecule capable of binding to the assessed analyte.

2. The dip-stick device of claim 1, wherein said device has three planar longitudinal faces, each such face having affixed thereto one of said first and second porous carriers, wherein the first porous carrier affixed to a first such face comprises a detectably labeled detector molecule capable of binding to an assessed analyte whose presence is characteristic of a Gram positive bacteria;
wherein the first porous carrier affixed to a second such face comprises a detectably labeled detector molecule capable of binding to an assessed analyte whose presence is characteristic of a Gram negative bacteria;
wherein the first porous carrier affixed to a third such face comprises a detectably labeled detector molecule capable of binding to an assessed analyte whose presence is characteristic of a viral pathogen.

3. The dip-stick device of claim 2, wherein said detectably labeled detector molecule capable of binding to an assessed analyte characteristic of a Gram positive bacteria is melanin.

4. The dip-stick device of claim 2, wherein said detectably labeled detector molecule capable of binding to an assessed analyte characteristic of a Gram negative bacteria is boc-Leu-Gly-Arg-paranitroaniline.

5. The dip-stick device of claim 2, wherein said detectably labeled detector molecule capable of binding to an assessed analyte characteristic of a viral pathogen is an antibody that immunospecifically reacts with a non-polio enterovirus, a paramyxovirus, an arbovirus, a herpes virus, a lymphocytic choriomeningitis virus, an adenovirus, a measles virus, or a human immunodeficiency virus.

6. The dip-stick device of claim 1, wherein said detectable label is an enzyme, and said dip-stick device additionally comprises a chromogenic substrate for such enzyme.

7. The dip-stick device of claim 2, wherein said detectable label is an enzyme, and said dip-stick device additionally comprises a chromogenic substrate for such enzyme.

8. The dip-stick device of claim 3, wherein said detectable label is an enzyme, and said dip-stick device additionally comprises a chromogenic substrate for such enzyme.

9. The dip-stick device of claim 4, wherein said detectable label is an enzyme, and said dip-stick device additionally comprises a chromogenic substrate for such enzyme.

10. The dip-stick device of claim 5, wherein said detectable label is an enzyme, and said dip-stick device additionally comprises a chromogenic substrate for such enzyme.

11. A method for conducting the simultaneous immunochromatographic analysis of two or more assessed analytes potentially contained in a fluid sample, wherein said method comprises the steps:
(A) Incubating a biological fluid in the presence of a device that comprises a solid support possessing three or more planar longitudinal faces, and at least one first and one second porous carrier affixed to at least one face thereof, said first and second porous carriers of each face being in fluid contact with one another, but spatially distinct from each other; wherein for each longitudinal face of said device having affixed carriers, said first porous carrier comprises a detectably labeled detector molecule capable of binding to one of said analytes to be assessed and the second such porous carrier contains an immobilized, but unlabeled capture molecule capable of binding to the assessed analyte;
(B) Permitting molecules of assessed analyte, if present, to migrate into said first porous carrier and to react with, or bind to, detectably labeled detector molecule present therein;
(C) Permitting molecules of assessed analyte, if present, that have reacted with, or bound to, said detectably labeled detector molecule to migrate into said second porous carrier and become immobilized to said capture molecule;
(D) Detecting whether detectably labeled detector molecules are immobilized to said immobilized capture molecules of said second porous carrier;
wherein detection of detectably labeled detector molecules immobilized to said immobilized capture molecules of said second porous carrier is indicative of the presence of said assessed molecule in said biological fluid.

12. The method of claim 11, wherein said device has three planar longitudinal faces, each such face having affixed thereto one of said first and second porous carriers, wherein the first porous carrier affixed to a first such face comprises a detectably labeled detector molecule capable of binding to an assessed analyte whose presence is characteristic of a Gram positive bacteria;
wherein the first porous carrier affixed to a second such face comprises a detectably labeled detector molecule capable of binding to an assessed analyte whose presence is characteristic of a Gram negative bacteria;
wherein the first porous carrier affixed to a third such face comprises a detectably labeled detector molecule capable of binding to an assessed analyte whose presence is characteristic of a viral pathogen.

13. The method of claim 12, wherein said detectably labeled detector molecule capable of binding to an assessed analyte characteristic of a Gram positive bacteria is melanin.

14. The method of claim 12, wherein said detectably labeled detector molecule capable of binding to an assessed analyte characteristic of a Gram negative bacteria is boc-Leu-Gly-Arg-paranitroaniline.

15. The method of claim 12, wherein said detectably labeled detector molecule is capable of binding to an assessed analyte characteristic of a viral pathogen is an antibody that immunospecifically reacts with a non-polio enterovirus, a paramyxovirus, an arbovirus, a herpes virus, a lymphocytic choriomeningitis virus, an adenovirus, a measles virus, or a human immunodeficiency virus.

16. The method of claim 11, wherein said detectable label is an enzyme, and said dip-stick device additionally comprises a chromogenic substrate for such enzyme.

17. The method of claim 12, wherein said detectable label is an enzyme, and said dip-stick device additionally comprises a chromogenic substrate for such enzyme.

18. The method of claim 13, wherein said detectable label is an enzyme, and said dip-stick device additionally comprises a chromogenic substrate for such enzyme.

19. The method of claim 14, wherein said detectable label is an enzyme, and said dip-stick device additionally comprises a chromogenic substrate for such enzyme.

20. The method of claim 15, wherein said detectable label is an enzyme, and said dip-stick device additionally comprises a chromogenic substrate for such enzyme.
